(19) United States
(12) Patent Application Publication
(10) Pub. No.: US 2008/0108855 A1
(43) Pub. Date: May 8, 2008

(54) PREPARATION OF HIGH ASSAY DECABROMODIPHENYLALKANE

(75) Inventors: Arthur G. Mack, Prairieville, LA (US); Saadat Hussain, Baton Rouge, LA (US)

Correspondence Address: McGLINCHEY STAFFORD, PLLC, 4703 BLUEBONNET BLVD, BATON ROUGE, LA, 70809, US

(73) Assignee: ALBEMARLE CORPORATION, Baton Rouge, LA (US)

(21) Appl. No.: 11/840,307

(22) Filed: Aug. 17, 2007

Related U.S. Application Data
(60) Provisional application No. 60/864,800, filed on Nov. 8, 2006.

(51) Int. Cl. C07C 17/093

(52) U.S. Cl. 570/206

(57) ABSTRACT

A process for producing reaction-derived decabromodiphenylalkane product, especially decabromodiphenylethane product, of high assay is described. Diphenylalkane, preferably diphenylethane, is brominated by introducing the selected diphenylalkane in the form of a mist or spray into a reaction zone containing a reaction mixture having a liquid phase comprised of excess bromine and Lewis acid bromination catalyst, so that mist or spray of diphenylalkane descends onto and contacts the liquid phase of the reaction mixture and bromination of the diphenylalkane occurs to produce reaction-derived decabromodiphenylalkane product of high assay. Preferably, the reaction mixture is maintained under reflux and optionally is also stirred.

### REFERENCE TO RELATED APPLICATION

This application claims the benefit and priority of U.S. Provisional Application No. 60/864,800, filed Nov. 8, 2006, the disclosure of which is incorporated herein by reference.

### TECHNICAL FIELD

This invention relates to the preparation of high assay decabromodiphenylalkane products such as decabromodiphenylethane products.

### BACKGROUND

Decabromodiphenylethane (DBDPE) is a time-proven flame retardant for use in many flammable macromolecular materials, e.g. thermoplastics, thermosets, cellulosic materials and back coating applications.

DBDPE is presently sold as a powder derived from the bromination of 1,2-diphenylethane (DPE). Among prior processes for effecting such bromination are the bromination processes described in U.S. Pat. Nos. 6,518,468; 6,958,423; 6,603,049; 6,768,033; and 6,974,887. While it has been possible in the past to produce very high assay DBDPE, this has not been accomplished on a consistent basis. Accordingly, it would be desirable if process technology could be provided that would enable the production of high assay DBDPE on a consistent basis.

### BRIEF SUMMARY OF THE INVENTION

This invention is deemed to enable production of high assay decabromodiphenylalkane products without recourse to recrystallization or chromatographic purification steps or any other subsequent procedure to remove or that removes nonabromodiphenylalkane from decabromodiphenylalkane such as decabromodiphenylethane. In addition, this invention is deemed to enable production of high assay DBDPE on a consistent basis.

Among the embodiments of this invention is a process for producing a reaction-derived decabromodiphenylalkane product of high assay, which process comprises introducing diphenylalkane in the form of a mist or spray into a reaction zone containing a reaction mixture having a liquid-phase comprised of bromine and a catalytic quantity of a Lewis acid bromination catalyst, so that mist or spray of diphenylalkane descends onto the liquid-phase of said reaction mixture.

In a preferred embodiment, the above process is conducted by refluxing the reaction mixture. Alternatively, or in addition, the reaction mixture is agitated by means of suitable mechanical apparatus such as mechanical stirring equipment. If the reaction mixture is not being refluxed, it is desirable to ensure that the reaction mixture is agitated at least during the time the mist or spray is being introduced into the reaction zone containing the reaction mixture, and preferably throughout substantially the entire reaction period.

In preferred embodiments, the diphenylalkane as fed into the reaction zone above the reaction mixture is diphenylethane whereby the reaction-derived product is high as say or high assay decabromodiphenylethane.

The above and other embodiments and features of this invention will be still further apparent from the ensuing description and appended claims.

### FURTHER DETAILED DESCRIPTION

It can be seen from the above that in the processes of this invention diphenylalkane is brominated in a reaction mixture having a liquid-phase comprised of bromine, and preferably containing an excess of refluxing bromine and typically Lewis acid bromination catalyst. An important feature of the invention is that the diphenylalkane is introduced into the reaction zone above the agitated or refluxing reaction mixture, and is introduced in the form of a mist or spray, preferably a fine spray. By “fine spray” is meant a spray in which droplets issue from the spraying device (e.g., one or more nozzles or spray heads), rather than a liquid stream. In the practice in this invention, when a spray is used as distinguished from a mist, the finer the droplets, the better.

In this connection, since the diphenylalkane is introduced into the reaction zone in the form of a mist or spray, a dip tube is not needed for the feeding of the diphenylalkane.

Without being bound to theoretical considerations, it is believed that the processes of this invention enable formation of high assay decabromodiphenylalkanes, especially decabromodiphenylethane, because of the rapid reaction which ensues when a mist or fine droplets of diphenylalkane, especially diphenylethane, descend onto and come in contact with the upper surfaces of the reaction mixture containing excess liquid bromine. The rapidity of the surface-contact-initiated bromination reaction, particularly when the reaction mixture is undergoing reflux, is deemed to be sufficient to result in formation of the decabromodiphenylalkane before any significant quantity of lower brominated species such as octobromodiphenylalkane and/or nonabromodiphenylalkane are able to separate from the liquid reaction mixture in the form of precipitates.

As noted above, the process technology of this invention is deemed applicable to the bromination of diphenylalkanes, i.e., compounds which can be depicted by the formula:

Ph-R-Ph

where Ph is a phenyl group and R is a straight chain alkylene group containing in the range of 1 to about 10 carbon atoms, preferably 1 or 2 carbon atoms, and more preferably the alkylene group has 2 carbon atoms (i.e., this more preferred reactant is 1,2-diphenylethane which is more commonly known as diphenylethane). Non-limiting examples of 1,2-diphenylalkanes which may be used as reactants in the processes of this invention include diphenylmethane, 1,3-diphenylpropane, 1,4-diphenylbutane, 1,3-diphenyl(2-methylpropane), 1,5-diphenylpentane, 1,6-diphenylhexane, 1,5-diphenyl(3-methylpentane), 1,4-diphenyl(2-methylpentane) and analogous compounds.

As used herein including the claims:

1) The term “reaction-derived” means that the composition of the product is reaction determined and not the result of use of downstream purification techniques, such as recrystallization or chromatography, or like procedures that can affect the chemical composition of the product. Adding water or an aqueous base such as sodium hydroxide to the reaction mixture to inactivate the catalyst, and washing away of non-chemically bound impurities by use of aqueous washes such as with water or dilute aqueous bases are not excluded by the term “reaction-derived”. In other words, the products are directly produced in the synthesis process without use of any subsequent procedure to remove or that removes nonabromodiphenylalkane from decabromodiphenylalkane.

2) The term “high assay” especially as applied to decabromodiphenylethane means that the reaction-derived DBDPE product comprises more than 97% of DBDPE with the balance consisting essentially of octabromodiphenyl ethane (Br₈DPE) and/or nonabromodiphenyl ethane (Br₉DPE) with the amount of Br₈DPE being less than the amount of Br₉DPE. Preferred reaction-derived DBDPE product comprises at least 98% of DBDPE and more preferred reaction-derived DBDPE product comprises at least 99% DBDPE, in both cases, with the balance consisting essentially of Br₈DPE and Br₉DPE, again with the amount of Br₉DPE exceeding the amount of Br₈DPE. Still more preferred are reaction-derived DBDPE products comprising at least 99.5% of DBDPE.

For the purposes of this invention, unless otherwise indicated, the % values given for DBDPE and nonabromodiphenyl ethane are to be understood as being the area % values that are derived from gas chromatography analysis. A procedure for conducting such analyses is presented hereinafter.

For convenience, the ensuing description will refer more specifically to bromination of diphenylethane. It is to be understood, however, that the principles apply to bromination of diphenylalkanes, and that the reaction conditions can be generally applied to the bromination of other diphenylalkanes.

The bromination process can be conducted either as a batch process or as a continuous process. Operation as a batch process is usually simpler and thus is preferred. In general, the duration of the feeding period in a batch process is inversely related to the temperature at which the refluxing is occurring. In other words, the higher the temperature, the shorter can be the feed time. When operating as a continuous process, the duration of the average residence time in the reactor is inversely related to the temperature at which the refluxing is occurring. Also, desirably, in a continuous process the rate of feed to the reactor and the rate of removal of the first reaction mixture from the reactor in a continuous process should be maintained such that the quantity of reaction mixture within the reactor remains substantially constant.

Increased reaction pressure tends to increase the extent of bromination. Nevertheless, pursuant to this invention, it is possible to operate at atmospheric, subatmospheric and/or at superatmospheric pressures in the range of about 1 to about 50 psig (ca. 1.08×10⁵ to 4.46×10⁵ Pa). However, the pressure is preferably no more than autogenous pressure in a closed reaction system.

The length of the feeding period in a batch process or of the residence time in a continuous process is temperature dependent. Thus, when the feeding time is taking place at a bromine refluxing temperature in the range of about 57° C. to about 60° C., the reaction time will typically be longer than when the bromination is occurring at a higher temperature. Thus, in carrying out a process of this invention, if the temperature-dependent period has not already been determined for the particular operation, a few laboratory experiments should be conducted for optimization purposes. Generally speaking, the feeding time of the mist or fine spray of diphenylethane should be in the range of about 1 to about 6 hours.

The coproduct in the reaction, hydrogen bromide, is typically released in part in the form of a vapor. For reasons of economy of operation it is desirable to recover the coproduct hydrogen bromide such as by passing the vapors into a scrubbing system in which the hydrogen bromide is converted either to hydrobromic acid using water as the scrubbing liquid, or into a hydrobromic acid salt using an aqueous solution of metal base such as aqueous sodium hydroxide as the scrubbing liquid.

This invention is deemed to enable the preparation of high assay DBDPE products that are derived from the bromination of diphenyl ethane. Such products can be said to be “reaction-derived” since they are reaction determined and not the result of use of downstream purification techniques, such as recrystallization, chromatography, or like procedures. In other words, the products of high as say are directly produced in the synthesis proces s apart from use of subsequent purification procedures that remove nonabromodiphenyl ethane from the decabromodiphenyl ethane product.

In the embodiments of this invention, 1,2-diphenylethane (also called dibenzyl or bibenzyl) is used. The term “diphenylethane” as used throughout this document means 1,2-diphenylethane unless otherwise noted. The DPE can be fed as solids, but preferably the feed is in molten form or as a solution in a solvent such as methylene bromide, chloroform, bromochloromethane, ethylene dichloride, or ethylene dibromide. Mixtures of such solvents can be used. To prevent freeze up in the feed conduit, DPE is desirably fed at a temperature of in the range of at least about 56° C. to about 80° C. Higher temperatures can be used if desired.

Excess bromine is used in the Lewis acid catalyzed bromination reaction. Typically, the reaction mixture will contain in the range of at least about 14 moles of bromine per mole of DPE to be fed thereto, and preferably, the reaction mixture contains in the range of about 16 to about 25 moles of bromine per mole of DPE to be fed thereto. It is possible to use more than 25 moles bromine per mole of DPE but this offers no advantage.

Typically the refluxing temperature of bromine at atmospheric or slightly elevated pressures is in the range of about 57 to about 59° C. but when operating at higher elevated pressures it is desirable to operate at somewhat higher temperatures in order to maintain a refluxing condition.

Various iron and/or aluminum Lewis acids can be added to the bromine and/or to the reaction mixture to serve as the bromination catalyst. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in a liquid phase of refluxing bromine. For example, one or more of the chlorine atoms of the aluminum chloride may possibly be replaced by bromine atoms. Other chemical changes are also possible. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

After all DPE is added, the reaction mixture can be kept at reflux for a suitable period of time to ensure completion of the perbromination to DBDPE. A period of up to about 3 hours is typically used.

Termination of the bromination reaction is typically effected by deactivating the catalyst with water and/or an aqueous base such as a solution of sodium hydroxide or potassium hydroxide.

The product formed in the bromination reaction is typically in the form of solid particles. These can be readily recovered from the reaction mixture by use of centrifugation, filtration, decantation or like solids/liquid physical separation procedures. The separated product is typically washed with water or dilute aqueous bases in order to wash away non-chemically bound impurities.

In order to determine the composition of the brominated product formed in a process of this invention, a gas chromatographic procedure is used. The gas chromatography is conducted on a Hewlett-Packard 5890 Series II gas chromatograph (or equivalent) equipped with a flame ionization detector, a cool on-column temperature and pressure programmable inlet, and temperature programming capability. The column is a 12 AQ HT5 capillary column, 12 meter, 0.15μ film thickness, 0.53 mm diameter, available from SGE, Inc., part number 054657. Conditions are: detector temperature 350° C.; inlet temperature 70° C.; heating at 125° C./min to 350° C. and holding at 350° C. until the end of the run; helium carrier gas at 10 ml/min.; inlet pressure 4.0 psig (ca. 1.29×10⁵ Pa), increasing at 0.25 psi/min. to 9.0 psig (ca. 1.63×10⁵ Pa) and holding at 9.0 psig until the end of the run; oven temperature 60° C. with heating at 12° C./min. to 350° C. and holding for 10 min.; and injection mode of cool on-column. Samples are prepared by dissolving, with warming, 0.003 grams in 10 grams of dibromomethane and injection of 2 microliters of this solution. The integration of the peaks is carried out using Target Chromatography Analysis Software from Thru-Put Systems, Inc. However, other and commercially available software suitable for use in integrating the peaks of a chromatograph may be used. Thru-Put Systems, Inc. is currently owned by Thermo Lab Systems, whose address is 5750 Major Blvd., Suite 200, Orlando, Fla. 32819. The address of SGE, Incorporated is 2007 Kramer Lane, Austin, Tex. 78758.

The decabromodiphenylalkane products formed in processes of this invention are white or slightly off-white in color. White color is advantageous as it simplifies the end-users task of insuring consistency of color in the articles that are flame retarded with such products.

The decabromodiphenylalkane products formed in the processes of this invention may be used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The decabromodiphenylalkane products formed in a process of this invention can also be used in textile applications, such as in latex-based back coatings.

The amount of a decabromodiphenylalkane product formed pursuant to this invention used in a formulation will be that quantity needed to obtain the flame retardancy sought. In general, the formulation and resultant product may contain from about 1 to about 30 wt %, preferably from about 5 to about 25 wt % of decabromodiphenylalkane product of this invention. Masterbatches of polymer containing decabromodiphenylalkane, which are blended with additional amounts of substrate polymer, typically contain even higher concentrations of decabromodiphenylalkane, e.g., up to 50 wt % or more.

It is advantageous to use the DBDPE products formed pursuant to this invention in combination with antimony-based synergists, e.g. Sb₂O₃. Such use is conventionally practiced in all DBDPE applications. Generally, the DBDPE products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the DBDPE products of this invention, e.g., plasticizers, antioxidants, fillers, pigments, UV stabilizers, etc.

Thermoplastic articles formed from formulations containing a thermoplastic polymer and DBDPE product of this invention can be produced conventionally, e.g., by injection molding, extrusion molding, compression molding, and the like. Blow molding may also be appropriate in certain cases.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

Except as may be expressly otherwise indicated, the article “a” or “an” if and as used herein is not intended to limit, and should not be construed as limiting, a claim to a single element to which the article refers. Rather, the article “a” or “an” if and as used herein is intended to cover one or more such elements, unless the text expressly indicates otherwise.

Each and every patent or publication referred to in any portion of this specification is incorporated in toto into this disclosure by reference, as if fully set forth herein.

That which is claimed is:

1. A process for preparing reaction-derived decabromodiphenylalkane of high assay, which process comprises introducing diphenylalkane in the form of a mist or spray into a reaction zone containing a reaction mixture having a liquid phase comprised of excess bromine and Lewis acid bromination catalyst, so that mist or spray of diphenylalkane descends onto the liquid phase of said reaction mixture and bromination of the diphenylalkane occurs to produce decabromodiphenylalkane.

2. A process as in claim 1 further comprising agitating said reaction mixture at least during a part of the time the mist or spray is being introduced into the reaction zone.

3. A process as in claim 1 further comprising maintaining the reaction mixture at one or more reflux temperatures at least during a part of the time the mist or spray is being introduced into the reaction zone.

4. A process as in claim 3 further comprising agitating said reaction mixture at least during a part of the time the mist or spray is being introduced into the reaction zone.

5. A process as in claim 1 wherein the bromination catalyst in the form introduced into the reaction mixture is an aluminum trihalide in which the halogen atoms are chlorine and/or bromine.

6. A process as in any of claims 1-5 wherein said diphenylalkane is diphenylethane.
